(19) United States
(12) Patent Application Publication
(10) Pub. No.: US 2007/0042972 A1
(43) Pub. Date: Feb. 22, 2007

(54) Compositions and methods for optimizing exercise recovery

(76) Inventors: Kenneth H. McKeever, Jackson, NJ (US); William C. Franke, New Brunswick, NJ (US); Robert Rosen, Monroe Township, NY (US); Sharon Rosen, Monroe Township, NY (US); Jennifer Streltsova, Neptune, NJ (US); Nettie Liburt, Orient, NY (US)

Correspondence Address: JONES DAY, 222 EAST 41ST ST, NEW YORK, NY, 10017, US

(21) Appl. No.: 11/439,718

(22) Filed: May 23, 2006

Related U.S. Application Data
(60) Provisional application No. 60/684,336, filed on May 24, 2005.

(51) Int. Cl. A61K 31/7048; A61K 31/353

(52) U.S. Cl. 514/27; 514/456

(57) ABSTRACT

The present invention provides methods for decreasing post-exercise recovery time in a subject using compositions comprising one or more polymethoxylated flavones (PMFs). In preferred embodiments, the composition is an orange peel extract as described herein. In certain embodiments, post-exercise recovery time is the time for a subject's post-exercise oxygen consumption (VO₂) level to return to a pre-exercise VO₂ level.

This application claims the benefit of U.S. Provisional Application No. 60/684,336, filed May 24, 2005, the contents of which are incorporated herein by reference in its entirety.

This invention was made with government support under contract number DAAD 16-02-C-005 awarded by the U.S. Army Natick Soldier System Center. The United States Government has certain rights in the invention.

### 1. TECHNICAL FIELD

The present invention relates to compositions and methods relating to use of orange peel extracts and polymethoxylated flavone compounds found in orange peels in producing beneficial exercise recovery results.

### 2. BACKGROUND

Exercise is generally accepted as a means to obtain cardiovascular health, weight control, and a subjective sense of well-being. Discomfort associated with exercise experienced as various physiological symptoms often deters individuals from participating in exercise programs. Moreover, some individuals may even avoid everyday activities such as walking or climbing stairs due to the physical exertion required to undertake the activity.

While formulations for reducing severity or time period of discomfort associated with exercise are highly desirable, such formulations should be safe and, preferably, health-promoting. In particular, it is desirable that such formulations be easily obtained, inexpensive and preferably from a natural source.

### 3. SUMMARY

In one aspect, the present invention provides methods for decreasing post-exercise recovery time in a subject. In certain embodiments, the subject is a mammal, more preferably a human. In particular, the methods provided comprise administering to the subject an effective amount of a composition comprising one or more polymethoxylated flavones (PMFs) to decrease the time the subject requires to recover from exercising.

In certain embodiments of the methods provided, the decrease in post-recovery time is between about 10 second to about 10 minutes, more typically between about 25 seconds to about 120 seconds.

In certain embodiments, the methods provided comprise administering an amount of a composition comprising one or more PMFs to the subject immediately prior to the subject beginning exercise, wherein the amount of the composition is effective to reduce the time taken for the subject's post-exercise oxygen consumption (VO₂) level to return to a pre-exercise basal VO₂ level.

In certain embodiments, “immediately” in this context refers to about 120, about 90, about 60, about 50, about 40, about 30, about 20, about 10, about 5 minutes or less prior to the subject beginning to exercise.

In some embodiments, the methods provided comprise administering an amount of a composition comprising one or more PMFs to the subject during exercise, wherein the composition is effective to reduce the time taken for the subject's post-exercise oxygen consumption (VO₂) level to return to a pre-exercise basal VO₂ level.

In some embodiments, the methods provided comprise administering an amount of a composition comprising one or more PMFs to the subject, wherein the amount of the composition is effective to complete the fast component of the return of the subject's post-exercise oxygen consumption (VO₂) level towards a pre-exercise basal VO₂ level.

In certain embodiments, methods for decreasing the time for a subject who has ceased exercising to have their post-exercise oxygen consumption (VO2) return to a pre-exercise basal VO₂ value are provided comprising administering a composition to the subject wherein the composition comprises a PMF fraction and a non-PMF fraction. Typically, the PMF fraction comprises one or more PMFs.

In some embodiments, the composition is orally administered to the subject.

In certain embodiments, the methods provide for optimizing post-exercise recovery in a subject comprising administering a composition to the subject prior to or during an exercise performed by the subject, wherein the composition comprises a non-PMF fraction and a PMF fraction comprising one or more PMFs, and wherein the composition is administered in an amount effective to decrease the time for the subject's post-exercise oxygen consumption (VO₂) level to return to a pre-exercise basal VO₂ level, thereby optimizing post-exercise recovery in the subject.

In another aspect, the present invention provides methods for increasing a subject's endurance for continued exercising or delaying fatigue in the subject while exercising comprising administering to the subject an amount of a composition comprising a non-PMF fraction and a PMF fraction comprising one or more PMFs to increase, wherein the amount of the composition administered is effective for increasing the subject's endurance for continued exercising or for delaying fatigue in the subject while exercising.

In yet another aspect, the present invention provides methods for reducing muscular soreness associated with exercise in a subject comprising administering an effect amount of a composition comprising one or more PMFs to the subject prior to exercising, during exercising, or after exercising by the subject.

In one aspect, the present invention provides methods for increasing a subject's exercise performance. In particular, in some embodiments, the methods provided comprise administering to the subject an amount of a composition comprising a PMF fraction and a non-PMF fraction, wherein the PMF fraction comprises one or more PMFs, and wherein the amount of composition administered is effective to increase the exercise performance of the subject.

In some embodiments, an increase in exercise performance is an increase in running speed or distance run by the subject.

In some embodiments, an increase in exercise performance is a delay in time to fatigue while the subject exercises.

In some embodiments, an increase in exercise performance is a reduction in lactic acid concentration that would otherwise occur in the absence of administering the composition according to the invention.

In some embodiments, an increase in exercise performance is an increase in number of repetitions the subject is able to do while weight lifting.

In some embodiments, an increase in exercise performance is the optimization of fat catabolism or heart rate while the subject exercises.

A composition in accordance with the invention can comprise one PMF or a plurality of PMFs. In general, the composition is not a natural source, such as, for instance, an orange peel.

In certain embodiments, the composition comprises about 25% (w/w) (dry weight) to about 75% (w/w) (dry weight) of a PMF fraction. In some embodiments, the PMF fraction is in a range from about 0.5% (w/w) to about 5% (w/w), from about 1% (w/w) to about 10% (w/w), from about 10% (w/w) to about 20% (w/w), from about 20% (w/w) to about 30% (w/w), from about 30% (w/w) to about 40% (w/w), from about 40% (w/w) to about 50% (w/w), from about 50% (w/w) to about 60% (w/w), from about 60% (w/w) to about 70% (w/w), from about 70% (w/w) to about 80% (w/w), or from about 80% (w/w) to about 98% (w/w).

In some embodiments of the methods provided, the composition comprises a PMF fraction comprising at least one, at least two, at least three, at least four, at least five, at least six, at least seven, at least eight, at least nine, at least ten, at least eleven, at least twelve, at least thirteen or all of the PMFs selected from the group consisting of 5,6,7,3′,4′-pentamethoxyflavone(sinensetin); 5,6,7,8,3′,4′-hexamethoxyflavone(nobeletin); 5,6,7,8,4′-pentamethoxyflavone(tangeretin); 5-hydroxy-6,7,8,3′,4′-pentamethoxyflavone(auranetin); 5-hydroxy-7,8,3′,4′-methoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3 ,5,6,3′,4′-pentamethoxyflavone.

In some embodiments, the PMF fraction of the composition for use in the methods of the invention consists of one, two, three, four, five, six, seven, eight, nine, ten, eleven, twelve, thirteen or all of PMFs selected from the group consisting of 5,6,7,3′,4′-pentamethoxyflavone(sinensetin); 5,6,7,8,3′,4′-hexamethoxyflavone(nobeletin); 5,6,7,8,4′-pentamethoxyflavone(tangeretin); 5-hydroxy-6,7,8,3′,4′-pentamethoxyflavone(auranetin); 5-hydroxy-7,8,3′,4′-methoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In some embodiments, the composition for use in the methods provided consists essentially of a physiologically acceptable solvent, excipient or carrier and one or more of 5,6,7,3′,4′-pentamethoxyflavone(sinensetin); 5,6,7,8,3′,4′-hexamethoxyflavone(nobeletin); 5,6,7,8,4′-pentamethoxyflavone(tangeretin); 5-hydroxy-6,7,8,3′,4′-pentamethoxyflavone(auranetin); 5-hydroxy-7,8,3′,4′-methoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In some embodiments, the PMF fraction of composition comprises one or more PMFs selected from the group consisting of 3,5,6,7,8,3′,4′-heptamethoxyflavone, 5-hydroxy-6,7,8,3′,4′-pentamethoxyflavone, and 5,7-dihydroxy-6,8,3′,4′-tetramethoxyflavone.

In some embodiments, the composition for use in the methods of the invention comprises a mixture of PMFs wherein the concentration of a PMF in the composition is different from that in a natural source of the PMF or that the ratio of one PMF in the composition to that of another PMF in the composition is different from that in a natural source of the PMFs. Such a composition can be prepared, for example, by processing a natural source of PMFs, for instance an orange peel, such that at least one particular PMF has been selectively removed, retained or enriched. Alternatively, one or more isolated or synthesized PMF can be used to make such compositions or added to a processed form of a natural source of PMFs.

In some embodiments, the composition for use in the methods of the invention comprises an orange peel extract.

In some embodiments, a composition for use in the methods of the invention can be a nutraceutical composition comprising one or more PMFs and a food, food additive, dietary supplement or medical food.

Typically, the amount of the PMF fraction of the composition administered to a subject in the methods of the invention is from about 0.05 mg, about 0.1 mg, about 0.5 mg, about 1.0 mg, about 5 mg, about 10 mg, about 15 mg, about 20 mg, about 25 mg, about 30 mg, about 35 mg, about 40 mg, or about 50 mg, to about 75 mg, about 100 mg, about 200 mg, about 250 mg, about 300 mg, about 400 mg, about 500 mg, about 600 mg, about 700 mg, about 750 mg, about 800 mg, about 900 mg, about 1 g, about 2 g, about 3 g, or about 5 g per day.

In some embodiments, the composition administered in the methods of the invention is administered via a buccal, nasal, oral, parenteral, rectal, sublingual, topical or transdermal route of administration. In certain embodiments, the composition is orally administered in an aqueous liquid form. In some embodiments wherein the composition is orally administered in an aqueous liquid form, the composition further comprises a mixing agent, blending agent or emulsifier, preferably lecithin.

In some embodiments, the composition administered in the methods of the invention is administered in an aerosol, chewable bar, bulk or loose dry form, capsule, cream, drink, elixir, emulsion, fluid, gel, granule, chewable gum, lotion, lozenge, ointment, paste, patch, pellet, powder, solution, spray, suppository, suspension, syrup, tablet, tea, tincture, vapor or wafer.

In certain embodiments of the methods provided, a composition comprising one or more PMFs and a bioactive ingredient (wherein the bioactive ingredient is not a PMF) is administered to the subject.

In certain embodiments, a “bioactive ingredient” in this context refers to any agent (not including a PMF) helpful in optimizing exercise performance including, for example, water, metabolites or precursors thereof, electrolytes, energy providing agents or catalysts to assist in obtaining energy, stimulants (for example, ephedrine, caffeine and the like), anti-inflammatory agents, and so forth without limitation. For example, in some embodiments, a “bioactive ingredient” can be a carbohydrate, monosaccharide, starch, pentose, protein, amino acid, polypeptide, triglyceride, fatty acid, vitamin or a mineral.

In some embodiments, the bioactive ingredient is ginger extract.

In yet another aspect, the invention provides a composition comprising a ginger extract for use in the methods as described herein.

### 4. BRIEF DESCRIPTION OF THE DRAWINGS

FIG. 1 provides the pharmacokinetics of the removal of a composition comprising a polymethoxylated flavone fraction from a horse administered with the composition.

FIG. 2 provides the recovery time observed in horses treated with water or orange peel extract and subjected to a graded exercise test indicating that a significant (p<0.05) difference between groups exists.

FIG. 3 provides the observed levels of aerobic capacity, i.e., maximal oxygen consumption (VO₂ₘₐₓ), observed in horses treated with water or orange peel extract and subjected to a graded exercise test.

FIG. 4 provides the observed levels of respiratory exchange ratio observed in horses treated with water or orange peel extract and subjected to a graded exercise test.

FIG. 5 provides the average run time observed in horses treated with water or orange peel extract and subjected to a graded exercise test.

FIG. 6 provides the plasma IFN-γ concentrations observed in horses treated with water or orange peel extract and subjected to a graded exercise test.

FIG. 7 provides the plasma TNF-α concentrations observed in horses treated with water or orange peel extract and subjected to a graded exercise test.

FIG. 8 provides (A) VO₂ₘₐₓ, (B) run-time to fatigue, and (C) cardiovascular recovery time in horses treated with cranberry (black bars), ginger (light bars), and water groups (grey bars). Asterisk (*) denotes a significant (p<0.05) difference between groups.

### 5. TERMINOLOGY

The term “about” as used herein refers to a value that is no more than 10% above or below the value being modified by the term. For example, the term “about 5 minutes” means a range of from 4.5 minutes to 5.5 minutes.

As used herein, the term “composition” is meant to encompass pharmaceutical compositions, physiologically acceptable compositions and nutraceutical compositions. It will be understood that where a component, for example, a polymethoxylated flavone (PMF), in a “composition” also occurs in a natural source (for instance, orange peel), the term “composition” does not include the natural source (for instance, orange peel) of the component, but can, in certain embodiments, encompass a physically or chemically modified or processed form of the natural source, such as an extract of the natural source.

The term “effective amount” as used herein refers to the amount of a compound or composition that is sufficient to produce a desirable or beneficial effect when administered to a subject. In certain embodiments, an “effective amount” of a compound or composition decreases post-exercise recovery time when administered to a subject. In some embodiments, an “effective amount” of a compound or composition increases a subject's endurance for continued exercising when administered to the subject. In some embodiments, an “effective amount” of a compound or composition reduces muscular soreness associated with exercise in a subject.

“Exercise” and “exercising” as used herein, refer to any physical activities by a subject that produces a peak oxygen consumption (“peak VO₂”), i.e., non-plateau phase, or maximal oxygen consumption (“VO₂ₘₐₓ”), i.e., plateau phase, in the subject in comparison to the subject's VO₂ prior to onset of the activity (“basal VO₂”). Upon cessation of the physical activity by the subject, the subject's peak VO₂ or VO₂ₘₐₓ moves toward or returns to the basal VO₂.

The term “fatigue,” as used herein, refers to a subject's inability to maintain a consistent level of physical activity or exercise. In certain embodiments, “fatigue” as used herein is meant to refer to the depletion of energy reserves necessary to maintain the consistent level of exercise, and/or the buildup of toxic metabolites in the subject, and the like, but is not due to lack of sleep, metabolic disease or illness.

The term “isolated,” when used in context of a compound or composition that can be obtained from a natural source, refers to a compound or composition that is separated from one or more components from its natural source. Natural sources can be a fungus, plant or animal or a natural and unaltered product produced by a fungus, plant or animal including bark, blood, cytosol, leaf, milk, mucous, peel, plasma, resin, rind, sap, sputum, stem, sweat, urine, and so forth. For example, a natural source can be an orange peel. Thus, an “isolated” compound or composition is in a form such that its concentration or purity is greater than that in its natural source. For example, in certain embodiments, an “isolated” compound or composition can be obtained by purifying or partially purifying the compound or composition from a natural source. In some embodiments, an “isolated” compound or composition is obtained in vitro in a synthetic, biosynthetic or semisynthetic organic chemical reaction mixture.

As used herein, the term “optimizing” refers to the beneficial effects that a subject derives when the methods provided herein are practiced on the subject. In certain embodiments, a subject is administered a composition as described herein to “optimize” post-exercise recovery so that the subject is able to engage in physical activity sooner than when not administered the compositions as described herein. In some embodiments, a subject is administered a composition as described herein to “optimize” post-exercise recovery so as to prevent or reduce time of muscular soreness experienced by the subject that would occur in the absence of administering the compositions as described herein. Typically, muscular soreness can be experienced by a subject one to three days following exercise, especially if the subject is unfit or unaccustomed to the type or intensity of exercise performed.

The terms “performance” and “exercise performance” refers to controlled movements by a subject that that can be maintained for the duration of an exercise to achieve a desired result of strength, speed, stamina, power, precision or metabolic output. In certain embodiments, an increase in performance can be measured, for example, as faster speed, as increased stamina, as higher power output, and so forth. Thus, in some embodiments, an increase in performance is an increase in number of repetitions or in mass lifted for a subject while weight lifting before reaching fatigue. In some embodiments, an increase in performance is an increase in velocity or in distance that a subject propels herself or himself, for example, while running, swimming or cycling before reaching fatigue. In some embodiments, an increase in performance is an increase in the maintenance of precision in motor movements such as throwing or catching, etc., while exercising before reaching fatigue. In some embodiments, an increase in performance can be measured in terms of physiological parameters, for example, as increased fat metabolism, optimal lactic acid metabolism, optimal heart rate, and so forth.

The term “polymethoxylated flavone” or “PMF” means, unless otherwise indicated, a compound having the formula 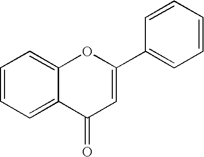
wherein at least one carbon, preferably two or more carbons, in the formula are attached to a —OCH₃ group (in place of one or more hydrogen atoms, not depicted in the formula) as valency permits. Optionally, substituents, such as, for example, hydroxyl, halide, monosaccharide, or other groups, may be substituted onto one or more carbons not substituted with a methoxy group. For example, a “hydroxylated PMF” is a PMF that comprises one or more hydroxyl groups attached to a carbon not substituted with a methoxy group. A “non-hydroxylated PMF” is a PMF that contains no hydroxyl groups.

“Recovery time from exercise” and “post-exercise recovery time” as used herein, refer to the time for a subject's peak VO₂ or VO₂ₘₐₓ to return to the subject's basal VO₂ after cessation of exercise by the subject. Without intending to be bound by any theory or mechanism, the return of a subject's peak VO₂ or VO₂ₘₐₓ to tha basal VO₂ is typically characterized by a fast phase, a slow phase, in certain instances, an ultra-slow phase. See Gaesser & Brooks (1984) Med. Sci. Sports Exerc. 16:29-43, incorporated herein by reference. In certain embodiments, “post-exercise recovery time” is the duration of the fast phase component. In certain embodiments, “post-exercise recovery time” is the duration of the fast phase component in addition to any slow phase components, if any, over which a subject's peak VO₂ or VO₂ₘₐₓ returns basal VO₂.

“Reducing muscular soreness” as used herein refers to a lessening or decrease in the severity of muscular soreness experienced by a subject brought about by exercise. In certain embodiments, “reducing muscular soreness” refers to the decrease in time that the subject experiences muscular soreness brought about by exercise.

“Solvate” refers to a compound, e.g., a PMF, that further includes a stoichiometric or non-stoichiometric amount of a solvent bound by non-covalent intermolecular forces. Where the solvent is water, the solvate is a hydrate.

As used herein, the terms “subject” and “patient” are used interchangeably. The terms “subject” and “subjects” refer to an animal, preferably a mammal including a non-primate and a primate (e.g., a monkey such as a chimpanzee, and a human), and more preferably a human. The term “animal” also includes, but is not limited to, companion animals such as cats and dogs; zoo animals; wild animals; farm or sport animals such as ruminants, non-ruminants, livestock and fowl (e.g., horses, cattle, sheep, pigs, turkeys, ducks, and chickens) including any animals and breeds of animals (e.g., greyhounds) used in racing; and laboratory animals, such as rodents (e.g., mice, rats), rabbits, and guinea pigs, as well as animals that are cloned or modified, either genetically or otherwise (e.g., transgenic animals).

### 6. DETAILED DESCRIPTION

As described in the Examples section below, a composition comprising a PMF fraction was discovered by empirical methods to have properties of reducing postexercise recovery times in exhaustive exercise in horses. Methods for using a composition comprising a PMF fraction are described in Section 6.1. PMF-containing compositions and methods for their preparation are described in Section 6.2.

6.1. Methods for Using PMF Compositions

The present invention provides methods for optimizing performance of, and/or recovery from, physical activity, e.g., exercise. Such methods are beneficial, for example, to subjects exercising to a state of fatigue. In some embodiments, the subject is a trained subject, such as an athlete. In some embodiments, the subject is an untrained subject, for example, a subject that leads a sedentary lifestyle or is a non-athlete.

In one aspect, the present invention provides methods for decreasing post-exercise recovery time in a subject in need thereof comprising administering to the subject an amount of a composition comprising a PMF fraction and a non-PMF fraction, wherein the PMF fraction comprises one or more polymethoxylated flavones (PMFs) and wherein the amount of the composition administered is effective to decrease the time the subject requires to recover from exercising.

Without intending to be bound by any particular theory or mechanism, post-exerecise recovery time can lasts seconds, typically tens of seconds, minutes, or hours, even up to twelve, twenty-four, or more hours in some instances. In certain embodiments, the decrease in post-recovery time is between about 10 seconds to about 10 minutes, more typically between about 25 seconds to about 120 seconds. In some embodiments of the methods provided, the the decrease in post-recovery time is about 5 seconds to about 1 minute, about 1 minute to about 5 minutes, about 5 minutes to about 20 minutes, about 20 minutes to about 1 hour, about 1 hour to about 5 hours, or about 5 hours to about 12 hours.

In certain embodiments, the methods provided comprise administering an amount of a composition comprising a PMF fraction and a non-PMF fraction to the subject, wherein the amount of the composition is effective to reduce the time taken for the subject's post-exercise oxygen consumption (VO₂) level to return to a pre-exercise basal VO₂ level.

In some embodiments, the methods provided comprise administering an amount of a composition of the invention to the subject, wherein the amount of the composition is effective to complete the fast component of the return of the subject's post-exercise oxygen consumption (VO₂) level towards a pre-exercise basal VO₂ level.

In certain embodiments, methods for decreasing the time for a subject who has ceased exercising to have their post-exercise oxygen consumption (VO₂) return to a pre-exercise basal VO₂ value are provided comprising administering a composition of the invention to the subject.

In some embodiments, the composition is orally administered to the subject.

In certain embodiments of the methods provided, the composition of the invention is administered to the subject immediately prior to subject beginning exercise.

In certain embodiments, “immediately” in this context refers to about 120, about 90, about 60, about 50, about 40, about 30, about 20, about 10, about 5 minutes or less prior to the subject beginning to exercise.

In some embodiments of the methods provided, a composition according to the invention is administered to the subject during exercise.

In certain embodiments, the methods provide for optimizing post-exercise recovery in a subject comprising administering a composition of the invention to the subject prior to or during an exercise performed by the subject, wherein the composition is administered in an amount effective to decrease the time for the subject's post-exercise oxygen consumption (VO₂) level to return to a pre-exercise basal VO₂ level, thereby optimizing post-exercise recovery in the subject.

In another aspect, the present invention provides methods for increasing a subject's endurance for continued exercising or delaying fatigue in the subject while exercising comprising administering to the subject an amount of a composition of the invention, wherein the amount of the composition administered is effective for increasing the subject's endurance for continued exercising or for delaying fatigue in the subject while exercising.

In certain embodiments, methods are provided for increasing a subject's endurance for continued exercising comprising administering to the subject an effective amount of a composition comprising a PMF fraction and a non-PMF fraction to increase the subject's endurance for continued exercising, wherein the PMF fraction comprises one or more PMFs, and wherein the composition is administered prior to exercising, during exercising, or within about 20 minutes after exercising by the subject.

In yet another aspect, the present invention provides methods for reducing muscular soreness associated with exercise in a subject comprising administering an effective amount of a composition comprising one or more PMFs to the subject prior to exercising, during exercising, or after exercising by the subject.

In certain embodiements, methods are provided for reducing muscular soreness associated with exercise in a subject comprising administering an effect amount of a composition comprising a PMF fraction and a non-PMF fraction to the subject prior to exercising, during exercising, or within about 20 minutes after exercising by the subject, wherein the PMF fraction comprises one or more PMFs.

In certain embodiments, the composition according to the invention can be administered within about 1 minute, about 10 minutes, about 20 minutes, about 40 minutes, or about one hour or more after the subject has ceased exercising.

In one aspect, the present invention provides methods for increasing a subject's exercise performance. In particular, in some embodiments, the methods provided comprise administering to the subject an amount of a composition comprising a PMF fraction and a non-PMF fraction, wherein the PMF fraction comprises one or more PMFs, and wherein the amount of composition administered is effective to increase the exercise performance of the subject.

In some embodiments, methods are provided for increasing a subject's exercise performance comprising administering to the subject an amount of a composition comprising a PMF fraction and a non-PMF fraction, wherein the PMF fraction comprises one or more PMFs, wherein the amount of composition administered is effective to increase the exercise performance of the subject, and wherein the composition is administered prior to exercising, during exercising, or within about 20 minutes after exercising by the subject. In certain embodiments, the increase in exercise performance is increased time to fatigue while exercising. In certain embodiments, the increase in exercise performance is an increase in running time.

In some embodiments, an increase in exercise performance is an increase in running speed or distance run by the subject. For example, in some embodiments, an increase in the subject's running speed or distance run can be about 1%, about 2%, about 3%, about 4%, about 5%, about 6%, about 7%, about 8%, about 9%, or about 10%.

In some embodiments, an increase in exercise performance is a delay in time to fatigue while the subject exercises. For example, in some embodiments, the delay to fatigue can be about 1%, about 2%, about 3%, about 4%, about 5%, about 6%, about 7%, about 8%, about 9%, or about 10% longer than would otherwise occur in absence of being administered with a composition comprising a PMF fraction and a non-PMF fraction.

In some embodiments, an increase in exercise performance is a reduction in lactic acid concentration that would otherwise occur in the absence of administering the composition according to the invention. For example, in some embodiments, there can be about 1%, about 2%, about 3%, about 4%, about 5%, about 6%, about 7%, about 8%, about 9%, or about 10% reduction in lactic acid concentration in the subject.

In some embodiments, an increase in exercise performance is an increase in number of repetitions the subject is able to do while weight lifting. For example, in some embodiments, the subject can complete one, two or more additional repetitions than would otherwise be possible in the absence of being administered with the composition according to the invention.

In some embodiments, an increase in exercise performance is the optimization of fat catabolism or heart rate while the subject exercises.

In certain embodiments, the invention provides methods for method for increasing a subject's exercise performance comprising administering to the subject an amount of a composition comprising a ginger extract, wherein the amount of composition administered is effective to increase the exercise performance of the subject, and wherein the composition is administered prior to exercising, during exercising, or within about 20 minutes after exercising by the subject.

6.2. PMF-Containing Compositions and Methods for Their Preparation

Compositions for use in the methods of the invention typically comprise a PMF fraction and a non-PMF fraction. In certain embodiments, the PMF fraction comprises one or more PMFs.

In certain embodiments, PMFs can be isolated, e.g., extracted, from a natural source for inclusion in compositions for use in the methods of the invention. In some embodiments a composition is an extract from a natural source comprising a PMF fraction. In some embodiments, compositions for use in the methods of the invention comprise an extract from cold-pressed orange peel oil solids. Preferably, compositions for use in the instant methods comprises an extract from Valencia and Hamlin varieties of oranges. Solvents useful for preparing extracts of orange peel for use as compositions in the methods of the invention include.

In some embodiments, the composition for use in the methods of the invention comprises a mixture of PMFs wherein the concentration of a PMF in the composition is different from that in a natural source of the PMF.

In some embodiments, the composition for use in the methods of the invention comprises a PMF fraction wherein a ratio of one PMF in the fraction to that of another PMF in the fraction is different from that in a natural source of the PMFs.

In certain embodiments, PMFs can be obtained synthetically for inclusion into compositions for use in methods of the invention. PMFs can be synthesized using any synthetic or semisynthetic technique, without limitation. A general synthetic scheme for flavones can found, for example, in Cushman and Nagarathnam (1990) Tetrahedron Letters 31: 6497-6500.

In certain embodiments, a composition for use in the methods of the invention comprises about 25% (w/w) (dry weight) to about 75% (w/w) (dry weight) of a PMF fraction. In some embodiments, the PMF fraction is in a range from about 0.5% (w/w) to about 5% (w/w), from about 1% (w/w) to about 10% (w/w), from about 10% (w/w) to about 20% (w/w), from about 20% (w/w) to about 30% (w/w), from about 30% (w/w) to about 40% (w/w), from about 40% (w/w) to about 50% (w/w), from about 50% (w/w) to about 60% (w/w), from about 60% (w/w) to about 70% (w/w), from about 70% (w/w) to about 80% (w/w), or from about 80% (w/w) to about 98% (w/w).

In certain embodiments, a PMF fraction in a composition for use in the methods of the invention comprises at least one, at least two, at least three, at least four, at least five, at least six, at least seven, at least eight, at least nine, at least ten, at least eleven, at least twelve, at least thirteen or all of the PMFs selected from the group consisting of 5,6,7,3′,4′-pentamethoxyflavone(sinensetin); 5,6,7,8,3′,4′-hexamethoxyflavone(nobeletin); 5,6,7,8,4′-pentamethoxyflavone(tangeretin); 5-hydroxy-6,7,8,3′,4′-pentamethoxyflavone(auranetin); 5-hydroxy-7,8,3′,4′-methoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In some embodiments, the PMF fraction of composition comprises one or more PMFs selected from the group consisting of 3,5,6,7,8,3′,4′-heptamethoxyflavone, 5-hydroxy-6,7,8,3′,4′-pentamethoxyflavone, and 5,7-dihydroxy-6,8,3′,4′-tetramethoxyflavone.

In some embodiments, the PMF fraction of the composition for use in the methods of the invention consists of at least one, at least two, at least three, at least four, at least five, at least six, at least seven, at least eight, at least nine, at least ten, at least eleven, at least twelve, at least thirteen or all of the PMFs selected from the group consisting of 5,6,7,3′,4′-pentamethoxyflavone(sinensetin); 5,6,7,8,3′,4′-hexamethoxyflavone(nobeletin); 5,6,7,8,4′-pentamethoxyflavone(tangeretin); 5-hydroxy-6,7,8,3′,4′-pentamethoxyflavone(auranetin); 5-hydroxy-7,8,3′,4′-methoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In some embodiments, the composition for use in the methods provided consists essentially of a physiologically acceptable solvent, excipient or carrier and at least one, at least two, at least three, at least four, at least five, at least six, at least seven, at least eight, at least nine, at least ten, at least eleven, at least twelve, at least thirteen or all of the PMFs selected from the group consisting of 5,6,7,3′,4′-pentamethoxyflavone(sinensetin); 5,6,7,8,3′,4′-hexamethoxyflavone(nobeletin); 5,6,7,8,4′-pentamethoxyflavone(tangeretin); 5-hydroxy-6,7,8,3′,4′-pentamethoxyflavone(auranetin); 5-hydroxy-7,8,3′,4′-methoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In certain embodiments, the composition for use in the methods provided consists of a PMF fraction and one or more non-PMF fractions, where the non-PMF fractions comprise, for example, a physiologically acceptable solvent, excipient, carrier, coloring agent, flavorant, food additive, nutrient, vitamin, mineral, metabolite, emulsifier, stabilizer, electrolyte, and so forth (that is, components other than a PMF), and where the PMF fraction is enriched for hydroxylated PMFs. The term “enriched,” as used herein in connection to a “hydroxylated PMF-enriched” fraction, encompasses a PMF fraction wherein hydroxylated PMFs in the fraction comprise at least 15% to about 95% of the total weight of the PMF fraction, and/or the proportion of hydroxylated PMFs to non-hydroxylated PMFs in the fraction is greater than the proportion of hydroxylated PMFs to non-hydroxylated PMFs found in natural sources that contain PMFs.

In certain embodiments, a “hydroxylated PMF-enriched” fraction in a composition comprises at least 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, or at least about 95% hydroxylated PMFs of the total weight of the PMF fraction.

In some embodiments, a composition for use in the methods provided comprises a hydroxylated PMF-enriched fraction and one or more non-PMF fractions, wherein the hydroxylated PMF-enriched PMF fraction consists of at least one, at least two, at least three, at least four, at least five, or more hydroxylated PMFs selected from the group of hydroxylated PMFs consisting of 3-hydroxy-5,6,7,4′-tetramethoxyflavone, 3-hydroxy-5,6,7,8,4′-pentamethoxyflavone, 3-hydroxy-5,6,7,8,3′,4′-hexamethoxyflavone, 5-hydroxy-3,6,7,8,3′,4′-hexamethoxyflavone, 5-hydroxy-3,7,8,3′,4′-pentamethoxyflavone, 5-hydroxy-3 ,7,3′,4′-tetramethoxyflavone, 5-hydroxy-6,7,8,3′,4′-pentamethoxyflavone, 5-hydroxy-6,7,8,3′,4′,5′-hexamethoxyflavone, 5-hydroxy-6,7,8,4′-tetramethoxyflavone, 5-hydroxy-6,7,4′-trimethoxyflavone, 5,3′-dihydroxy-6,7,8,4′-tetramethoxyflavone, 5-hydroxy 7,8,3′,4′ tetramethoxyflavone, 5,7-dihydroxy-6,8,3′,4′ tetramethoxyflavone, 7-hydroxy 3,5,6,8,3′,4′ hexamethoxyflavone, 7-hydroxy 3,5,6,3′,4′ pentamethoxyflavone, 3′-hydroxy-5,6,7,4′-tetramethoxyflavone, 3′-hydroxy-5,6,7,8,4′-pentamethoxyflvone, 3′,4′-dihydroxy-5,6,7,8-tetramethoxyflavone, and 4′-hydroxy-5,6,7,8,3′-pentamethoxyflavone.

In certain embodiments of the methods provided, a composition comprising one or more PMFs and a bioactive ingredient (wherein the bioactive ingredient is not a PMF) is administered to the subject.

In certain embodiments, a “bioactive ingredient” in this context refers to any agent (not including a PMF) helpful in optimizing exercise performance including, for example, water, metabolites or precursors thereof, electrolytes, energy providing agents or catalysts to assist in obtaining energy, stimulants (for example, ephedrine, caffeine and the like), anti-inflammatory agents, and so forth without limitation. For example, in some embodiments, a “bioactive ingredient” can be a carbohydrate, monosaccharide, starch, pentose, protein, amino acid, polypeptide, triglyceride, fatty acid, vitamin or a mineral.

In some embodiments, the bioactive ingredient is ginger extract.

In yet another aspect, the invention provides a composition comprising a ginger extract for use in the methods as described herein.

In certain embodiments, a composition comprising a mixture of orange peel extract and ginger extracts can be used in the methods provided herein.

6.2.1. Nutraceutical Formulations

In certain embodiments, a composition for use in the methods of the invention can be a nutraceutical composition. As used herein, the term “nutraceutical composition” refers to a composition comprising a food, food additive, dietary supplement, medical food or food for special dietary use and a PMF fraction.

In some embodiments, a nutraceutical composition of the invention typically comprises one or more consumable vehicles, carriers, excipients, or fillers. The term “consumable” means generally suitable for, or is approved by a regulatory agency of the Federal or a state government for, consumption by animals, and more particularly by humans.

As used herein, “food” means any substance, whether processed, semi-processed, or raw, which is intended for consumption by animals including humans, but does not include cosmetics, tobacco products or substances used only as pharmaceuticals.

As used herein, the term “dietary supplement” means a product (other than tobacco) intended to supplement the diet. Typically, a dietary supplement is a product that is labeled as a dietary supplement and is not represented for use as a conventional food or as a sole item of a meal or the diet. A dietary supplement can typically comprises one or more of the following dietary ingredients: a vitamin; a mineral; an herb or other botanical; an amino acid; a dietary supplement used by man to supplement the diet by increasing the total dietary intake; or a concentrate, metabolite, constituent, extract, or a combination of any of the ingredients. A dietary supplement can be consumed by a subject independent of any food, unlike a food additive which is incorporated into a food during the processing, manufacture, preparation, or delivery of the food, or just prior to its consumption.

As used herein, the term “medical food” refers to a food which is formulated to be consumed or administered enterally under the supervision of a physician or veterinarian and which is intended for the specific dietary management of a disease or condition for which distinctive nutritional requirements, based on recognized scientific principles, are established by medical evaluation. Examples of medical foods include but are not limited to sole source nutrition products which are complete nutritional products used to replace all other food intake; oral rehydration solutions for use in replacing fluids and electrolytes lost following diarrhea or vomiting; modular nutrient products containing specially selected components not intended to be complete nutritional sources but designed for the management of specific diseases and which have associated claims to effectiveness either direct or implied; and products intended for use in dietary management of inborn errors of metabolism.

As used herein, the term “food for special dietary use” refers to a food which is represented to be used for at least one of the following: supplying a special dietary need that exists by reason of a physical, physiological, pathological, or other condition, including but not limited to the condition of disease, convalescence, pregnancy, lactation, infancy, allergic hypersensitivity to food, underweight, overweight, or the need to control the intake of sodium; supplying a vitamin, mineral, or other ingredient for use by man to supplement his diet by increasing the total dietary intake; and supplying a special dietary need by reason of being a food for use as the sole item of the diet.

The nutraceutical compositions for use in the methods of the invention can also include one or more other ingredients that impart additional healthful or medicinal benefits.

In some embodiments, a nutraceutical composition for use in the methods of the invention comprises a PMF fraction and one or more “Generally Regarded As Safe” (“GRAS”) substance(s). Many GRAS substances are known and are listed in the various sections of the regulations of the United States public health authority, 21 CFR 73, 74, 75, 172, 173, 182, 184 and 186, which are incorporated herein by reference in their entirety.

In certain embodiments, the meaning of the term “medical food”, “food for special dietary use”, “dietary supplement” or “food additive” is the meaning of those terms as defined by a regulatory agency of a state government or the federal government of the United States, including the United States Food and Drug Administration.

In certain embodiments, the nutraceutical compositions for use in the methods of the invention comprise from about 0.001% to about 90%, by weight of a PMF fraction. Other amounts of the combination that are also contemplated are from about 0.0075% to about 75%, about 0.005% to about 50%, about 0.01% to about 35%, 0.1% to about 20%, 0.1% to about 15%, 1% to about 10%, and 2% to about 7%, by weight of the PMF fraction.

In certain embodiments of the methods provided, the composition comprising one or more PMFs is administered to the subject as aqueous based beverage. Such beverages can further comprise physiologically acceptable additives such as suspending agents (e.g., sorbitol syrup, cellulose derivatives or hydrogenated edible fats); emulsifying agents (e.g., lecithin or acacia); and preservatives (e.g., methyl or propyl-p-hydroxybenzoates or sorbic acid). The preparations may also contain buffer salts, electrolytes, flavoring, coloring and sweetening agents as appropriate.

In some embodiments, the composition administered in the methods of the invention is administered in an aerosol, chewable bar, bulk or loose dry form, capsule, cream, drink, elixir, emulsion, fluid, gel, granule, chewable gum, powder, solution, spray, suspension, syrup, tablet, or tea.

In certain embodiments, the composition is administered in a drink or gel intended for optimizing exercise performance or for nourishing, replacing, replenishing, or recovering fluids, nutrients, calories, and the like as needed as a result of exercise. Sports drinks and gels are generally known and include, for example, GATORADE sports drink, GU energy gel, GU₂O sports drink, CRANK eGEL energy gel, CYTOMAX sports drink, POWERBAR PERFORM drink, ULTIMA REPLENISHER drink, SOBE SPORTS SYSTEM drink, ULTRAFUEL drink, SHAKELEE PHYSIQUE drink, ENDUROXR4 drink, HAMMER gel, CLIF SHOT gel, CARB-BOOM gel, ACCELERADE drink, ACCEL gel, and the like.

6.2.2. Pharmaceutical Formulations

In certain embodiments, pharmaceutical compositions comprising one or more PMFs and one or more physiologically acceptable carriers or excipients for use in accordance with the present invention may be formulated in conventional manner. Thus, the combination of one or more physiologically acceptable carriers or excipients and one or more PMFs and their physiologically acceptable salts and solvates may be formulated for administration by inhalation or insufflation (either through the mouth or the nose) oral, buccal, parenteral, rectal, or transdermal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the one or more PMFs.

For buccal administration the compositions may take the form of tablets or lozenges formulated in conventional manner.

For administration by inhalation, the pharmaceutical compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the one or more PMFs and a suitable powder base such as lactose or starch.

The pharmaceutical composition may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The pharmaceutical composition may also be formulated in rectal compositions such as suppositories or retention enemas, e.g., containing conventional suppository bases such as cocoa butter or other glycerides.

In addition to the formulations described previously, the pharmaceutical composition may also be formulated as a depot preparation. Such long acting formulations may be administered by implantation (for example subcutaneously or intramuscularly) or by intramuscular injection. Thus, for example, the complexes may be formulated with suitable polymeric or hydrophobic materials (for example as an emulsion in an acceptable oil) or ion exchange resins, or as sparingly soluble derivatives, for example, as a sparingly soluble salt.

The pharmaceutical composition may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

6.3. Administration of PMF-Containing Compositions

The amount of the composition to be administered to a subject in the methods of the invention, as well as the frequency of administration, will vary, for example, with the age, body weight, response and past medical history of the subject. Effective doses may be extrapolated from dose-response curves derived from in vitro or animal model test systems.

Generally, the active ingredient, i.e, the one or more PMFs, of the compositions used in methods of the invention are administered to a subject in amounts of about 0.001 μg/kg, about 0.005 μg/kg, about 0.01 μg/kg, about 0.05 μg/kg, about 0.1 μg/kg, about 0.5 μg/kg, about 1 μg/kg, about 5 μg/kg, about 10 μg/kg, about 50 μg/kg, about 100 μg/kg, about 500 μg/kg, about 1 mg/kg, about 5 mg/kg, about 10 mg/kg, about 25 mg/kg, about 50 mg/kg, about 67 mg/kg, about 75 mg/kg, or about 80 mg/kg, where the units refer to mass of the active ingredient per subject body weight (kg).

In certain embodiments, where the composition is to be administered to a subject, preferably a human, in the methods of the invention, the amount of the PMF fraction in the administered composition is from about 0.05 mg, about 0.1 mg, about 0.5 mg, about 1.0 mg, about 5 mg, about 10 mg, about 15 mg, about 20 mg, about 25 mg, about 30 mg, about 35 mg, about 40 mg, or about 50 mg, to about 75 mg, about 100 mg, about 200 mg, about 250 mg, about 300 mg, about 400 mg, about 500 mg, about 600 mg, about 700 mg, about 750 mg, about 800 mg, about 900 mg, about 1 g, about 2 g, about 3 g, about 5 g per, or about 10 g.

In some embodiments, the amount of PMF fraction to be administered in the composition is from about 50 mg to about 1 g.

In some embodiments, the amount of PMF fraction to be administered in the composition is from about 100 mg to about 400 mg.

In certain embodiments of the methods provided, the composition comprising one or more PMFs is administered to the subject prior to exercising. In some embodiments, the composition is administered within about 12 hours, about 6 hours, about 3 hours, about 2 hours, about 1 hours, about 30 minutes, about 15 minutes, or within about 1 minute before exercising.

In certain embodiments of the methods provided, the composition comprising one or more PMFs is administered to the subject while the subject is exercising.

In certain embodiments of the methods provided, preferably in embodiments of methods for reducing muscular soreness associated with exercise in a subject, the composition is administered to the subject after exercising. In some embodiments, the compositon is administered to the subject within about 1 minute, about 10 minutes, about 20 minutes, about 1 hour, about 3 hours, about 6 hours, about 12 hours, about 24 hours, about 36 hours or within about 48 hours after exercising.

The PMF containing composition can be administered by any suitable route that ensures bioavailability of the PMF fraction in the subject's circulation. Any route of administration that provides an effective amount of the PMF-containing composition can be used. In particular, the route of administration can be indicated by the type of formulation, e.g., nutraceutical or pharmaceutical composition, as described above.

In some embodiments, the composition administered in the methods of the invention is administered via a buccal, nasal, oral, parenteral, rectal, sublingual, topical or transdermal route of administration.

In preferable embodiments, the composition comprising one or more PMFs is orally administered. In some embodiments, the composition comprising one or more PMFs is orally administed in liquid form, preferably an aqueous liquid form.

### 7. EXAMPLES

The following exemplifies the use of compositions comprising a polymethoxylated flavone component for decreasing post-exercise recovery time in a subject.

7.1. Preparation of Extracts

7.1.1. Orange Peel Extract

Orange solids, including peels, a byproduct from the orange juice industry, were obtained from Valencia and Hamlin varieties of oranges. Orange peel oil was obtained by cold-pressing the peels. Orange peel oil contains about 0.4% of a PMF fraction, a 98% light volatile fraction and 2% residue. A separation process utilizing extraction with solvents followed by drying the extract was performed to yield an orange peel extract in powder form. The orange peel extract had a PMF fraction, representing approximately 30% (w/w) of the extract, and a non-PMF fraction. The non-PMF fraction contained little or no detectable limonene and was found to to contain waxes, unsaturated fatty acids and β-sitosterol. The PMFs were analyzed by reverse phase high performance liquid chromatography (HPLC) and normal phase HPLC.

Typically, for normal phase HPLC, a silica gel HPLC column (MacMod Analytical Co., Chadds Ford, Pa.) with dimensions of 4.6 mm i.d.×25 cm length, was utilized with 90% hexane/10% chloroform starting solvent. Runs were performed using a 10% to 90% chloroform gradient over 20 minutes, followed by another 20 minutes at 90% chloroform. Mass spectrometry (MS) was used in conjunction with HPLC to identify individual PMFs. Atmospheric pressure chemical ionization MS was used for molecular weight determination. Standards were obtained from the Florida Department of Citrus (Lakeland, Fla.). The orange peel extract powder comprised a mixture of various analogs of methoxylated flavonoids, including:
5,6,7,3′,4′-pentamethoxyflavone(sinensetin);
5,6,7,8,3′,4′-hexamethoxyflavone(nobeletin);
5,6,7,8,4′-pentamethoxyflavone(tangeretin);
5-hydroxy-6,7,8,3′,4′-pentamethoxyflavone(auranetin);
5-hydroxy-7,8,3′,4′-methoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

7.2. Administration of Orange Peel Extract to Horses

Healthy untrained Standardbred mares were used in the studies described below. Horse physiology is similar to that of humans in terms of (1) cardiovascular function, (2) metabolic and muscular response to exercise, (3) endocrine response to exercise, and (3) thermoregulation by sweating.

For pharmokinetics studies in horses, 35 grams of orange peel extract powder, prepared as described Section 7.1, dissolved in three liters of water was administered to the subjects. For administration to horses in the graded exercise test (GXT), 30 grams of orange peel extract powder dissolved into two liters of water was administered to the subjects as described below. The protocol to solubilze the orange peel extract powder included dissolving the orange peel extract powder in 100 mL of ethanol and adding 10 g lecithin. The mixture was brought to a boil and then slowly added to warm (120° F.) water under high shear conditions to form a pre-emulsion. The pre-emulsion was then dispersed in warm water under high shear conditions to make the desired final volume.

Orange peel extract dissolved in water was administered to horses by nasogastric tube.

7.3. Pharmacokinetics of Orange Peel Extact in Horses

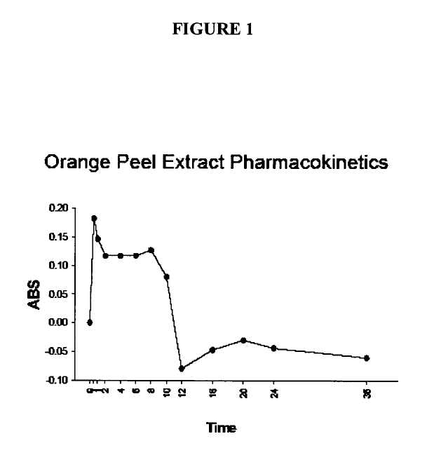

After administration of orange peel extract to horses as described above, blood samples were collected at 0.5, 1, 2, 4, 6, 8, 10, 12, 16, 20, 24 and 36 hours from the horses and subjected to HPLC/MS to observe orange peel extract component in plasma. Results are shown in FIG. 1.

Physiological observations of, e.g., temperature, respiration, heart rate, mucous membrane color, skin fold and overall appearance and behavior were noted.

7.4. Graded Exercise Test

7.4.1. Methods

Test Protocol: A graded exercise test (GXT) on a treadmill was utilized to study responses to exercise in horses treated with orange peel extract. A randomized crossover blind study was designed using six unfit mares randomly assigned to treatment with water or orange peel extract. The ages of the mares were 10±4 years and had an average mass of about 450 kg. The mares were housed in a pasture with water and pasture grazing provided ad libitum and fed approximately 6 kg/day alfalfa and grass hay, approximately 3 kg/day grain. The horses were accustomed to the lab and running on the treadmill prior to the start of the experiment.

Before each test run on the treadmill, horses were weighed and catheters (2-Angiocath, 14 gauge, Becton Dickson, Inc., Parsippany, N.H.; and a 7 French catheter introducer, Argon Medical, Athens, Tex.) were introduced percutaneously into the left and right jugular veins, respectively, using sterile techniques and local lidocaine anesthesia. Each horse was administered 30 g extract in 2 L water by nasogastric tube and left to stand quietly for one hour in its stall. The horses were then walked onto the treadmill where a thermister probe (Model # Bat-1, Physitemp Instruments, Clifton, N.J.) was inserted and positioned for measurement of core body temperature. A micromanometer catheter transducer (Millar Instruments, Houston, Tex.) was employed to measure pulmonary artery pressure, right ventricular pressure and heart rate. Verification of the position of the pressure-sensing catheters was performed before and after exercise by using the representative blood pressure waveforms recorded on the physiological recording system (Biopac, Santa Barbara, Calif.). The horses stood quietly for approximately 10-15 min. Fifteen minutes of hemodynamic data, standing calorimetry data, were obtained after which a baseline blood sample (10 ml), and a rectal temperature (YSI PRECISION 4000A Thermometer, YSI Inc., Yellow Springs, Ohio) were taken.

The graded exercise test protocol performed on a horse SATO I treadmill (Equine Dynamics, Inc., Lexington, Ky.) at a fixed 6% grade began with an initial speed of 4 m/s for 1 minute followed by a ramp up to 6 m/s for 1 minute with incremental 1 m/s increases in speed every minute thereafter until fatigue of the horse. Fatigue was identified as the point at which the horse could not keep up with the treadmill despite humane encouragement. At fatigue, the treadmill was stopped, and 5 minutes of post-exercise calorimetry and hemodynamic data were collected. One week was allowed between treadmill sessions.

Calorimetry: An indirect open-flow OXYMAX-XL calorimeter apparatus (Columbus Instruments, Inc., Columbus, Ohio) was used to measure oxygen consumption (VO₂) and carbon dioxide (VCO₂) continuously during the test and recorded at ten second intervals. The maximal oxygen uptake (VO₂ₘₐₓ), observed as the plateau in VO₂, was determined for each horse and the horse's velocity noted (V_{VO2max}). Recovery time was determined as the time taken for VO₂ to return to preexercise levels.

Blood Chemistry: Blood samples were taken prior to GXT, at the end of each one minute step of the GXT, and over a twenty four hour period after fatigue (i.e., at 2 min., 5 min., 30 min., 1 hr., 2 hr., 4 hr., and 24 hr. post-GXT). Blood parameters observed included hematocrit, total protein, and concentrations in plasma of lactate, creatine kinase and aspartate aminotransferase. Blood samples were placed into prechilled VACUTAINER tubes containing EDTA or sodium heparin (Becton Dickson, Inc., Franklin Lakes, N.J.) and were immediately placed on ice. Lactate concentrations were measured in triplicate using a lactate SPORT 1500 analyzer (YSI, Inc., Yellow Springs, Ohio). Hematocrit and plasma protein were measured in duplicate using the microhematocrit technique and refractometry. McKeever et al. (1998) Vet. J. 155:19-25. Creatine kinase and aspartate aminotransferase were measured using an enzymatic reaction assay (BMD Roche/Hitachi 747-100, High Technology, Inc., Walpole, Mass.).

Measurement of Cytokine Expression: Expression levels of interleukin 6 (IL-6), tissue necrosis factor alpha (TNF-α) and gamma interferon (IFN-γ) RNAs were determined in blood samples drawn by venipuncture from horses and collected into PAXGENE® tubes (Qiagen, Valencia, Calif.), prior to GXT, immediately post exercise, and at 0.5, 1, 2, 4, and 24 hs post exercise. Total RNA was isolated from the tubes using spin columns according to the manufacturer's instructions. RNA was quantitated using a BIOPHOTOMETER spectrophotometer (Eppendorf, Hamburg, Germany). In all cases, OD_{260/280} ratios were greater than 1.9 and RNA yields were greater than 50 μg/ml. One microgram of RNA was reverse transcribed into cDNA in an 80 μl reaction containing 20 units of AMV reverse transcriptase, 0.5 μg of oligo dT primers, 40 units of RNAsin and 5 mM MgCl₂ (Promega, Madison, Wis.). Cytokine-specific cDNA was then amplified and quantitated by “real-time” polymerase chain reaction (PCR) (ABI Sysytems 7500 Sequence Detection System, Foster City, Calif.) using the Taq thermostable DNA polymerase and primers based on known sequences for equine cytokines and β-actin. See Swiderski et al. (1999) J. Immunol. Methods 222:155-69. Specific primers and FAM-labeled probes for each cytokine and β-actin, provided as ASSAY-BY-DESIGN kits (ABI), were added to 25 μl reactions in 96-well microplates containing the Taq polymerase (12.5 μl of UNIVERSAL MASTER MIX reaction buffer, ABI) and 5 μl of cDNA. The following PCR conditions were employed; 95° C. for 10 mins followed by 40 cycles of 95° C. for 15 secs and 60° C. for 60 secs, as recommended by the manufacturer.

Differences in RNA isolation and cDNA construction between samples were corrected using β-actin as an internal control for each sample. Relative differences in cytokine mRNA expression resulting from exercise were determined by relative quantification. Relative quantitation provides accurate comparison between the initial levels of target cDNA in a sample without requiring that the exact copy number be determined. See Livak & Schmittgen (2001) Methods 25:402-408. The pre-exercise samples were selected as the calibrator and the change in cytokine gene expression post-expression relative to the calibrator was then determined for each sample.

Statistical Analysis: Results are expressed as means±standard error of the mean (SEM). For comparison by group and time a two-way ANOVA for repeated measures was used with the a priori level of statistical significance set at P<0.05. Post hoc differences were determined using the Tukey test, and correlation coefficients were derived using the Pearson product moment (Sigma Stat 2.0; SPSS Inc., Chicago, Ill.).

7.4.2. Results

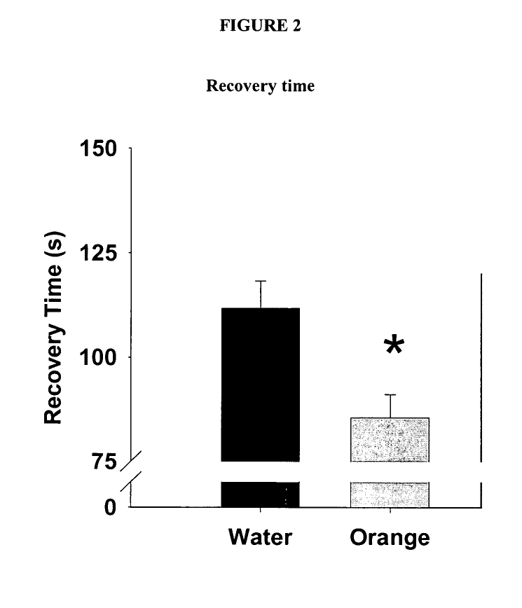

FIG. 2 depicts a comparison of the VO₂ recovery time, showing that post-exercise recovery time was significantly reduced in the group treated with orange peel extract as compared to groups treated with water.

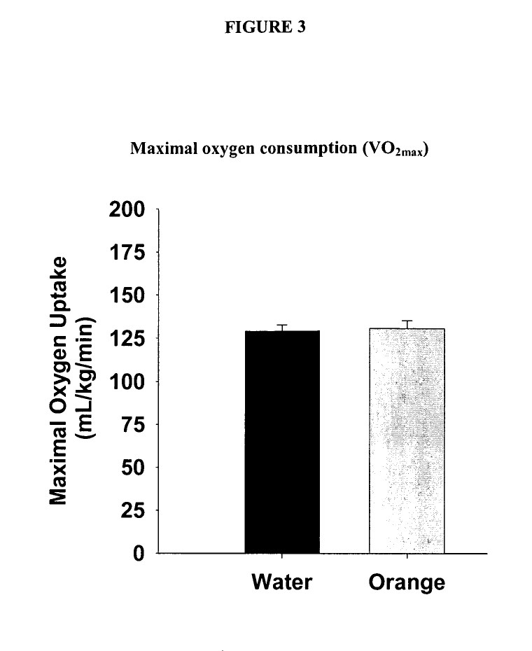

Results of the aerobic capacity, i.e., maximal oxygen consumption (VO₂ₘₐₓ), for groups treated with water or orange peel extract are shown in FIG. 3. No differences (p>0.05) in maximal aerobic capacity between the treatment group and control group were noted.

No differences (p>0.05) were observed between the treatment group and control group in core body temperature, rectal temperature, heart rate or hemactocrit.

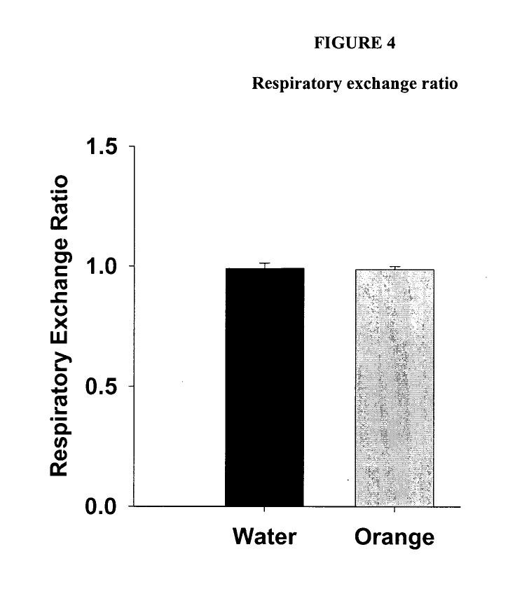

A comparison of the respiratory exchange ratio in each of the groups is shown in FIG. 4. No differences (p>0.05) in respiratory exchange ratio between the treatment and control groups were noted.

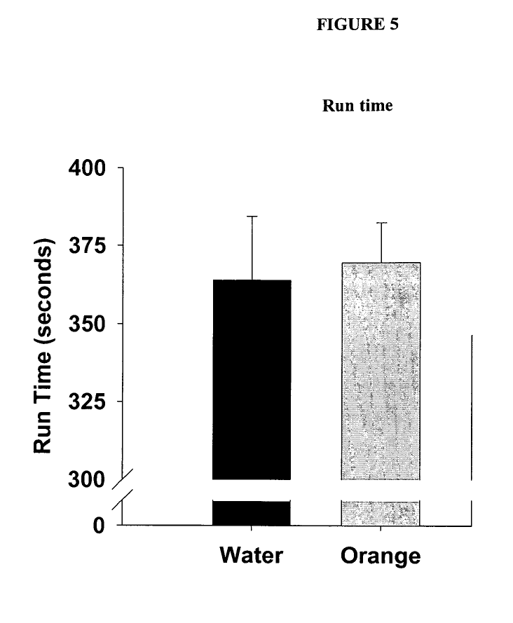

Results of the average run time to fatigue per treatment group are depicted in FIG. 5.

Changes in creatine kinase concentrations before (approximately 240 IU/L) and after exercising (approximately 350 IU/L at two and four hours after exercise) were observed in horses administered with orange peel extract and in horses administered with water. Creatine kinase levels returned to nearly pre-exercise levels in both groups by 24 hours post-exercise. No significant differences between horses treated with orange peel extract and those treated with water were observed in terms of plasma lactate, creatine kinase and aspartate aminotransferase concentrations were observed.

Plasma volume was reduced during exercise, believed to be a shift in fluid compartmentalization as reflected, for example, in a change in plasma protein concentration. The total plasma protein concentration, initially near 7.7 g/dL pre-exercise, increased to a maximal concentration of approximately 10 g/dL near the point of fatigue in horses treated with water. In comparison, total plasma protein concentration in horses treated with orange peel extract was observed to rise from approximately 7.5 g/dL pre-exercise to a maximal of 8.2 g/dL over the course of the exercise. Thus, orange peel extract appeared to reduce the decrease in plasma volume during exercise.

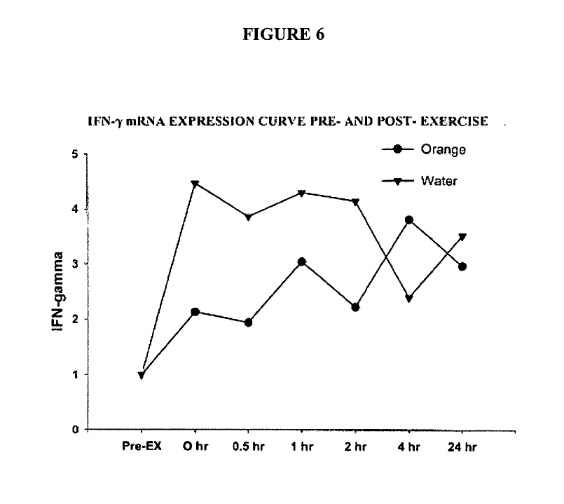

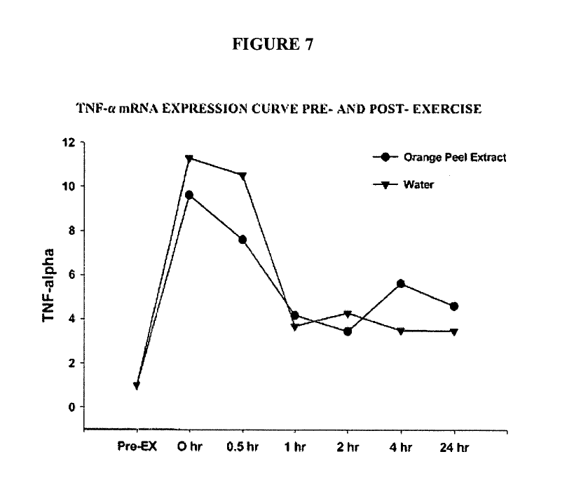

Data that reflecting cytokine levels as markers for inflammation indicate that exercise increases IFN-γ mRNA expression (FIG. 6) and TNF-α mRNA (FIG. 7), but not IL-6 mRNA expression. Some differences in exercise-induced IFN-γ mRNA upregulation between horses treated with orange peel extract and those treated with water occurred in some of the earlier time points (FIG. 6). Slight or no differences in TNF-α and IL-6 mRNA expression levels were observed between horses treated with orange peel extract and with water.

7.5. Effects of Ginger Extract on Exercise Performance and Recovery

The purpose of this study was to investigate the effects of ginger (Zingiber officinale) and cranberry (Vaccinium macrocarpon) extracts on markers of performance (VO₂, hematocrit, plasma total protein), thermoregulation (core and rectal temperature) and muscle damage (creatine kinase, or CK, and aspartate aminotransferase, or AST) after an exhaustive bout of exercise in horses. The test protocol and methodology for obtaining physiological data were identical to that described above in Section 7.4 except that horses were treated with ginger extract, cranberry extract or water. In particular, nine unfit Standardbred mares completed 3 graded exercise tests (GXTs) in a randomized crossover design. Mares received a dose of either water (2 L), cranberry (˜30 g in 2 L water) or ginger (˜30g in 2 L water) extract approximately 1 hr prior to testing. Blood samples were taken prior to dosing (pre-ex), at the end of each increment on the treadmill, end of exercise, 2 min, 5 min, 30 min, 1 hr, 2 hr, 4 hr and 24 hr post-GXT. Plasma total protein and hematocrit values were analyzed immediately following the exercise test.

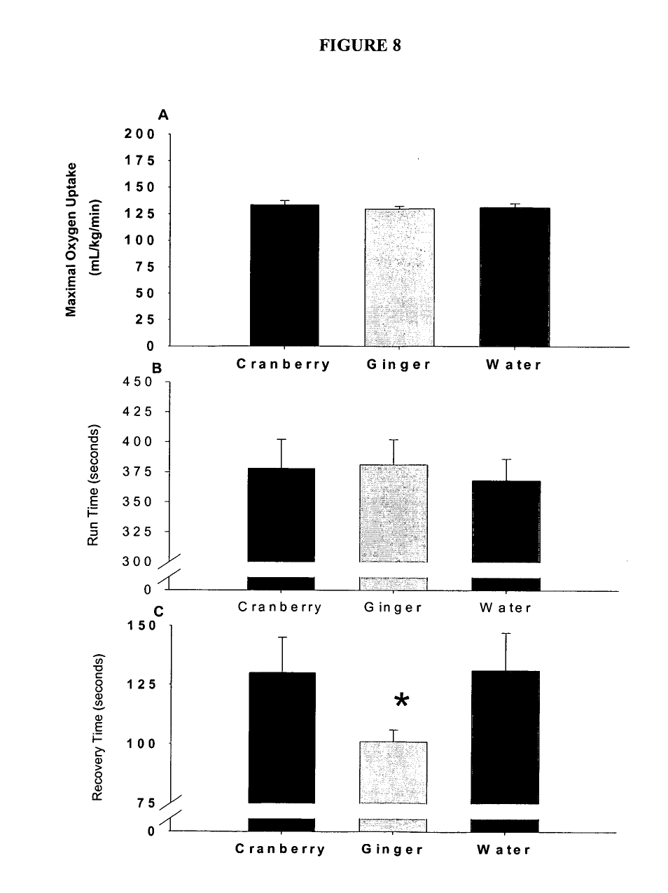

As shown in FIG. 8(C), ginger significantly lowered cardiovascular recovery time. No effect of treatment (p>0.05) was seen on VO₂ₘₐₓ (FIG. 8(A)), hematocrit or plasma total protein. Results suggest that ginger extract reduces cardiovascular recovery time in horses completing a exhaustive bout of exercise.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed:

1. A method for decreasing post-exercise recovery time in a subject comprising administering to the subject an effective amount of a composition comprising a PMF fraction and a non-PMF fraction to decrease the time a subject requires to recover from exercising, wherein the PMF fraction comprises one or more PMFs, and wherein the composition is administered prior to exercising, during exercising, or within about 20 minutes after exercising by the subject.

2. The method of claim 1, wherein the decrease in post-exercise recovery time is between about 25 seconds to about 120 seconds.

3. The method of claim 1, wherein the post-exercise recovery time is the time taken for the subject's post-exercise oxygen consumption (VO₂) level to return to a pre-exercise basal VO₂ level.

4. The method of claim 1, wherein the post-exercise recovery time is the time taken to complete the fast component of the return of the subject's post-exercise oxygen consumption (VO₂) level towards a pre-exercise basal VO₂ level.

5. The method of claim 1 for decreasing the time for a subject who has ceased exercising to have their post-exercise oxygen consumption (VO₂) return to a pre-exercise basal VO₂ value comprising administering to the subject an effective amount of a composition comprising a PMF fraction and a non-PMF fraction wherein the PMF fraction comprises one or more PMFs and wherein the composition is orally administered to the subject prior to when the subject ceases exercising, whereby the time taken for the subject's post-exercise VO₂ to return to a pre-exercise basal VO₂ value is shorter than the time for the subject's post-exercise VO₂ to return to a pre-exercise basal VO₂ value when not administered with the composition.

6. The method of claim 1, wherein the one or more PMFs are selected from the group consisting of
5,6,7,3′,4′-pentamethoxyflavone(sinensetin);
5,6,7,8,3′,4′-hexamethoxyflavone(nobeletin);
5,6,7,8,4′-pentamethoxyflavone(tangeretin);
5-hydroxy-6,7,8,3′,4′-pentamethoxyflavone(auranetin);
5-hydroxy-7,8,3′,4′-methoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

7. The method of claim 6, wherein the PMF fraction of the composition consists essentially of
5,6,7,3′,4′-pentamethoxyflavone(sinensetin);
5,6,7,8,3′,4′-hexamethoxyflavone(nobeletin);
5,6,7,8,4′-pentamethoxyflavone(tangeretin);
5-hydroxy-6,7,8,3′,4′-pentamethoxyflavone(auranetin);
5-hydroxy-7,8,3′,4′-methoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; or
7-hydroxy-3,5,6,3′,4′-pentamethoxyflavone.

8. The method of claim 1, wherein the PMF fraction consists of one or more PMF selected from the group consisting of
5,6,7,3′,4′-pentamethoxyflavone(sinensetin);
5,6,7,8,3′,4′-hexamethoxyflavone(nobeletin);
5,6,7,8,4′-pentamethoxyflavone(tangeretin);
5-hydroxy-6,7,8,3′,4′-pentamethoxyflavone(auranetin);
5-hydroxy-7,8,3′,4′-methoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

9. The method of any one of claim 1, wherein the one or more PMFs constitute an about 25% to about 75% w/w fraction of the composition.

10. The method of claim 9, wherein the composition is a food, food additive, dietary supplement, or medical food.

11. The claim 1, wherein the composition comprises a non-PMF fraction that comprises a physiologically acceptable carrier or excipient and a PMF fraction that comprises one or more PMFs selected from the group consisting of
5,6,7,3′,4′-pentamethoxyflavone(sinensetin);
5,6,7,8,3′,4′-hexamethoxyflavone(nobeletin);
5,6,7,8,4′-pentamethoxyflavone(tangeretin);
5-hydroxy-6,7,8,3′,4′-pentamethoxyflavone(auranetin);
5-hydroxy-7,8,3′,4′-methoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

12. The method of claim 1, wherein the composition comprises a non-PMF fraction that comprises a physiologically acceptable carrier or excipient and a PMF fraction that consists essentially of one or more PMFs selected from the group consisting of
5,6,7,3′,4′-pentamethoxyflavone(sinensetin);
5,6,7,8,3′,4′-hexamethoxyflavone(nobeletin);
5,6,7,8,4′-pentamethoxyflavone(tangeretin);
5-hydroxy-6,7,8,3′,4′-pentamethoxyflavone(auranetin);
5-hydroxy-7,8,3′,4′-methoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

13. The method of claim 1, wherein the composition comprises a non-PMF fraction that comprises a physiologically acceptable carrier or excipient and a PMF fraction that consists of one or more PMFs selected from the group consisting of
5,6,7,3′,4′-pentamethoxyflavone(sinensetin);
5,6,7,8,3′,4′-hexamethoxyflavone(nobeletin);
5,6,7,8,4′-pentamethoxyflavone(tangeretin);
5-hydroxy-6,7,8,3′,4′-pentamethoxyflavone(auranetin);
5-hydroxy-7,8,3′,4′-methoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

14. The method of claim 9, wherein the composition is orally administered to the subject.

15. The method of claim 14, wherein the composition is administered in a dry form.

16. The method of claim 14, wherein the composition is administered in a liquid form.

17. The method of claim 14, wherein the subject is a human.

18. The method of claim 14, wherein the subject is a horse.

19. The method of claim 14, wherein the subject is a dog.

20. The method of claim 17, wherein about 50 mg to about 1000 mg of the PMF fraction in the composition is administered to the subject.

21. The method of claim 1, wherein the non-PMF fraction comprises a bioactive ingredient.

22. The method of claim 21, wherein the bioactive ingredient is ginger extract.

23. The method of claim 1, wherein the composition is administered within about 2 hours, about 1 hour or within about 30 minutes before the subject exercises.

24. The method of claim 1, wherein the composition is administered while the subject exercises.

25. The method of claim 1, wherein the composition comprises molecules isolated from orange peels and from ginger.

26. A method for decreasing post-exercise recovery time in a subject comprising administering to the subject an effective amount of a composition comprising a ginger extract to decrease the time a subject requires to recover from exercising, wherein the composition is administered prior to exercising, during exercising, or within about 20 minutes after exercising by the subject.

27. The method of claim 26, wherein the ginger extract further comprises an orange peel extract.
